SEQUENCE LISTING (8 sequences)

SEQ ID NO: 1 - protein, 214 aa, Artificial Sequence [sequence omitted]

SEQ ID NO: 2 - protein, 448 aa, Artificial Sequence
  Tocilizumab heavy chain
  MOD_RES LOCATION: (1)..(1): Pyroglutamic Acid
XVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 10 aa, Artificial Sequence
  Tocilizumab CDR L1
RASQDISYLN

SEQ ID NO: 4 - protein, 7 aa, Artificial Sequence
  Tocilizumab CDR L2
YTSRLHS

SEQ ID NO: 5 - protein, 8 aa, Artificial Sequence
  Tocilizumab CDR L3
QGNTLPYT

SEQ ID NO: 6 - protein, 6 aa, Artificial Sequence
  Tocilizumab CDR H1
SDHAWS

SEQ ID NO: 7 - protein, 15 aa, Artificial Sequence
  Tocilizumab CDR H2
YISYSGITYNPSLKS

SEQ ID NO: 8 - protein, 9 aa, Artificial Sequence
  Tocilizumab CDR H3
SLARTAMDY

(19) United States
(12) Patent Application Publication
(10) Pub. No.: US 2023/0174656 A1
(43) Pub. Date: Jun. 8, 2023

(54) TOCILIZUMAB AND REMDESIVIR COMBINATION THERAPY FOR COVID-19 PNEUMONIA

(71) Applicant: Genentech, Inc., South San Francisco, CA (US)

(72) Inventors: Min Bao, Cupertino, CA (US); Larry Wilse Tsai, Hillsborough, CA (US); Mark Douglas Eisner, San Francisco, CA (US)

(73) Assignee: Genentech, Inc., South San Francisco, CA (US)

(21) Appl. No.: 17/906,912

(22) PCT Filed: Mar. 19, 2021
(86) PCT No.: PCT/US2021/023061

§ 371 Date: Sep. 21, 2022

Related U.S. Application Data
(60) Provisional application No. 62/993,589, filed on Mar. 23, 2020.
(60) Provisional application No. 63/011,889, filed on Apr. 17, 2020.

(51) Int. Cl. C07K 16/28; A61P 31/14; A61K 31/706; A61K 45/06

(52) U.S. Cl. CPC C07K 16/2866; A61K 31/706; A61K 45/06; A61P 31/14

(57) ABSTRACT

The application describes a method of treating viral pneumonia in a patient comprising administering an effective amount of a combination of tocilizumab and remdesiver to the patient.

### CROSS REFERENCE TO RELATED APPLICATIONS

This application cliams the benefit U.S. Provisional Application No. 62/993589, filed Mar. 23, 2020, and U.S. Provisional Application No. 63/011,889 filed on Apr. 17, 2020, which are incorpated by reference in entirety.

### SEQUENCE LISTING

The instant application contains a sequence listing submitted via efs-web and is hereby incorporated by reference in its entirety. Said ASCII copy, created Mar. 15, 2021, is named P36381WOSEQLIST.txt, and is 7,358 bytes in size.

### FIELD OF THE INVENTION

The invention concerns methods of treating pneumonia in patients with an IL6 antagonist. It includes methods for treating viral pneumonia, such as coronavirus pneumonia, and exemplified by COVID-19 pneumonia. In one embodiment, it concerns administering a weight-based intravenous dose of tocilizumab to the patient, wherein the weight-based dose is 8 mg/kg of tocilizumab. In one embodiment, IL-6 level has not been found to be elevated in the patient. Optionally, the method further comprises administering a second weight-based 8 mg/kg intravenous dose of tocilizumab to the patient 8-12 hours after the first dose (e.g. 8-11 hours after the first dose) or 8-24 hours after the first dose, wherein the patient experiences no improvement or ≥ one-category worsening on an ordinal scale of clinical status, following the first dose. In another embodiment, it concerns administering an IL6 antagonist (e.g. an IL6 receptor antibody such as tocilizumab) to a patient in an amount effective to achieve a greater improvement in clinical outcome than standard of care (SOC), e.g. as measured on an ordinal scale of clinical status, optionally in combination with other efficacy and safety outcomes as disclosed in more detail herein. In another embodiment, the invention concerns a method of treating acute respiratory distress syndrome (ARDS) in a patient who does not have elevated IL6 level comprising administering an IL6 antagonist (e.g. an IL6 receptor antibody such as tocilizumab) to the patient.

### BACKGROUND OF THE INVENTION

Interleukin-6 (IL-6) is a proinflammatory, multifunctional cytokine produced by a variety of cell types. IL-6 is involved in such diverse processes as T-cell activation, B-cell differentiation, induction of acute phase proteins, stimulation of hematopoietic precursor cell growth and differentiation, promotion of osteoclast differentiation from precursor cells, proliferation of hepatic, dermal and neural cells, bone metabolism, and lipid metabolism (Hirano T. Chem Immunol. 51: 153-180 (1992); Keller et al. Frontiers Biosci. 1: 340-357 (1996); Metzger et al. Am J Physiol Endocrinol Metab. 281: E597-E965 (2001); Tamura et al. Proc Natl Acad Sci USA. 90: 11924-11928 (1993); Taub R. JClin Invest 112: 978-980 (2003)). IL-6 has been implicated in the pathogenesis of a variety of diseases including autoimmune diseases, osteoporosis, neoplasia, and aging (Hirano, T. (1992), supra; and Keller et al., supra). IL-6 exerts its effects through a ligand-specific receptor (IL-6R) present both in soluble and membrane-expressed forms.

Elevated IL-6 levels have been reported in the serum and synovial fluid of rheumatoid arthritis (RA) patients, indicative of production of IL-6 by the synovium (Irano et al. EurJ Immunol. 18: 1797-1801 (1988); and Houssiau et al. Arthritis Rheum. 1988; 31:784-788 (1988)). IL-6 levels correlate with disease activity in RA (Hirano et al. (1988), supra), and clinical efficacy is accompanied by a reduction in serum IL-6 levels (Madhok et al. Arthritis Rheum. 33:S154. Abstract (1990)).

Tocilizumab (TCZ) is a recombinant humanized monoclonal antibody of the immunoglobulin IgG1 subclass which binds to human IL-6 receptor. Clinical efficacy and safety studies of intravenous (iv) TCZ have been completed or are conducted by Roche and Chugai in various disease areas, including adult-onset RA, systemic juvenile idiopathic arthritis (sJIA) and polyarticular juvenile idiopathic arthritis (pJIA).

Tocilizumab is approved in the United States for:
1. Rheumatoid Arthritis (RA): Adult patients with moderately to severely active rheumatoid arthritis who have had an inadequate response to one or more Disease-Modifying Anti-Rheumatic Drugs (DMARDs).
2. Giant Cell Arteritis (GCA): Adult patients with giant cell arteritis.
3. Polyarticular Juvenile Idiopathic Arthritis (pJIA): Patients 2 years of age and older with active polyarticular juvenile idiopathic arthritis.
4. Systemic Juvenile Idiopathic Arthritis (sJIA): Patients 2 years of age and older with active systemic juvenile idiopathic arthritis.
5. Cytokine Release Syndrome (CRS): Adults and pediatric patients 2 years of age and older with chimeric antigen receptor (CAR) T cell-induced severe or life-threatening cytokine release syndrome.

Coronaviruses (CoV) are positive-stranded RNA viruses with a crown-like appearance under an electron microscope due to the presence of spike glycoproteins on the envelope. They are a large family of viruses that cause illness ranging from the common cold to more severe diseases such as Middle East respiratory syndrome (MERS-CoV) and severe acute respiratory syndrome (SARS-CoV).

COVID-19, which is the acronym of “coronavirus disease 2019,” is caused by a new coronavirus strain that has not been previously identified in humans and was newly named on 11 Feb. 2020 by the World Health Organization (WHO). An epidemic of cases with unexplained lower respiratory tract infections was first detected in Wuhan, the largest metropolitan area in China’s Hubei province, and was reported to the WHO Country Office in China on Dec. 31, 2019. A pandemic was subsequently declared by the WHO on 11 Mar. 2020.

According to the WHO, as of 17 Mar. 2020 over 179,000 cases of COVID-19 were reported in over 100 countries worldwide, with over 7400 deaths. Up to ~20% of infected patients experienced complications related to a severe form of interstitial pneumonia, which may progress towards acute respiratory distress syndrome (ARDS) and/or multi organ failure (MOF) and death.

To date, there is no vaccine and no specific anti-viral medicine shown to be effective in preventing or treating COVID-19. Most patients with mild disease recover with symptomatic treatment and supportive care. However, those patients with more severe illness require hospitalization (WHO 2020).

CRS has been identified as a clinically significant, on-target, off-tumor side effect of the CAR T-cell therapies used for treatment of malignancies. Characteristics of CRS include fever, fatigue, headache, encephalopathy, hypotension, tachycardia, coagulopathy, nausea, capillary leak, and multi-organ dysfunction. The reported incidence of CRS after CAR T-cell therapy ranges from 50% to 100%, with 13% to 48% of patients experiencing the severe or life-threatening form. Serum levels of inflammatory cytokines are elevated, particularly interleukin-6 (IL-6). The severity of symptoms may correlate with the serum cytokine concentrations and the duration of exposure to the inflammatory cytokines.

On Aug. 30, 2017, the U.S. Food and Drug Administration approved tocilizumab (ACTEMRA®) for the treatment of severe or life-threatening CAR T cell-induced CRS in adults and in pediatric patients 2 years of age and older. The approved dose is 8 mg/kg for body weight ≥ 30 kg and 12 mg/kg for body weight < 30 kg. Up to three additional doses may be given if no improvement of sign/symptoms, and the interval between the subsequent doses should be at least 8 hours.

The approval of TCZ was based on a retrospective analysis of data for patients treated with TCZ who developed CRS after treatment with tisagenlecleucel (KYMRIAH®) or axicabtagene ciloleucel (YESCARTA®) in prospective clinical trials (Le et al. The Oncologist. 23:943-947 (2018)). Thirty-one out of the 45 patients (69%) from the CTL019 series achieved a response (defined as being afebrile and off vasopressors for at least 24 hours within 14 days of the first dose of TCZ (maximum up to two doses) and without use of additional treatment other than corticosteroids) within 14 days of the first dose of TCZ, and the median time from the first dose to response was 4 days. Eight of the 15 patients (53%) from the axicabtagene ciloleucel series achieved a response, and the median time to response was 4.5 days. The response rates were largely consistent among subgroups such as age group, sex, race, ethnicity, grade of CRS at first dose of TCZ, and duration of CRS prior to treatment with TCZ. There were no reports of adverse reactions attributable to TCZ.

Pharmacokinetic (PK) data were available for 27 patients after the first dose of TCZ and for 8 patients after a second dose of TCZ. Based on 131 PK observations, the geometric mean (% CV) maximum concentration of TCZ in the patients with CAR T cell induced, severe or life-threatening CRS was 99.5 µg/mL (36.8%) after the first infusion and 160.7 µg/mL (113.8%) after the second infusion. The PK modeling analysis showed that patients with CRS had a faster clearance of TCZ than healthy volunteers and other patient populations, and simulations showed that exposure was considered acceptable with up to four doses of TCZ at least 8 hours apart in patients with CRS.

TCZ is also approved for CAR-T induced severe or life-threatening CRA in European Union and certain other countries.

Physicians in China initiated the off-label usage of TCZ in the treatment of coronavirus (COVID-19) pneumonia. Based on the findings of an observational study of 21 COVID-19 patients treated with TCZ, an investigator-initiated randomized, open-label study (n = 188) was also initiated on 13 Feb. 2020.

On 3 Mar. 2020, TCZ was included in the Seventh Edition “Diagnosis and Treatment Protocol of COVID-19 Pneumonia” by the China National Health Commission as one treatment option for severe or critical forms of COVID-19 pneumonia. The Chinese CDC defined disease severity according to the following criteria:
1. Severe pneumonia: dyspnea, respiratory frequency ≥ 30/min, blood oxygen saturation (SpO₂) ≤ 93%, PaO2/FiO₂ ratio [the ratio between the blood pressure of the oxygen (partial pressure of oxygen, PaO2) and the percentage of oxygen supplied (fraction of inspired oxygen, FiO₂)] < 300 mmHg, and/or lung infiltrates > 50% within 24 to 48 hours; this occurred in 14% of cases.
2. Critical pneumonia: respiratory failure, septic shock, and/or multiple organ dysfunction (MOD) or failure (MOF); this occurred in 5% of cases (Wu et al. JAMA. doi:10.1001/jama.2020.2648 (2020)).

According to Section 10.3.7 of these Guidelines: “For patients with extensive lung lesions and severe patients, and laboratory testing of elevated IL-6 levels, tocilizumab treatment can be tried. The first dose is 4 to 8 mg/kg, the recommended dose is 400 mg, 0.9% saline is diluted to 100 ml, and the infusion time is more than 1 hour; if no clinical improvement in the signs and symptoms occurs after the first dose, it can be applied at the same dose as before more after 12 hours. The cumulative number of administrations is a maximum of 2 times, and the maximum single dose does not exceed 800 mg. Pay attention to hypersensitivity, and those with active infection such as tuberculosis are contraindicated.”

Based on the results of an initial 21-patient retrospective observational study in which patients with severe or critical coronavirus (COVID-19) pneumonia were treated with TCZ, a randomized, controlled trial (n = 188) has been initiated in the same population testing the same TCZ dose regimen and is currently ongoing with approximately 70 patients enrolled. Xu et al. Effective treatment of severe COVID-19 patients with tocilizumab. Submitted manuscript. [Resource on the internet]. 2020 [updated 5 Mar. 2020; cited 17 Mar. 2020]. Available from: http://www.chinaxiv.org/abs/202003.00026.

In February 2020, twenty-one patients with severe or critical COVID-19 pneumonia were treated with TCZ IV (400 mg) plus standard of care. The average age of the patients was 56.8 ± 16.5 years, ranging from 25 to 88 years. Seventeen patients (81.0%) were assessed as severe and four (19.0%) as critical. Most patients (85%) presented with lymphopenia. C-reactive protein (CRP) levels were increased in all 20 patients (mean, 75.06 ± 66.80 mg/L). The median procalcitonin (PCT) value was 0.33 ± 0.78 ng/mL, and only two of 20 patients (10.0%) presented with an abnormal value. Mean IL-6 level before TCZ was 132.38 ± 278.54 pg/mL (normal < 7 pg/mL).

Standard of care consisted of lopinavir, methylprednisolone, other symptom relievers, and oxygen therapy as recommended by the Diagnosis and Treatment Protocol for Novel Coronavirus Pneumonia (Sixth Edition). All 21 patients had received routine standard of care treatment for a week before deteriorating with sustained fever, hypoxemia, and chest CT image worsening.

Eighteen patients (85.7%) received TCZ once, and three patients (14.3%) had a second dose due to fever within 12 hours. According to the authors, after TCZ treatment, fever returned to normal and all other symptoms improved remarkably. Fifteen of the 20 patients (75.0%) had lowered their oxygen intake and one patient needed no oxygen therapy. CT scans showed significant remission of opacities in both lungs in 19/20 patients (90.5%) after treatment with TCZ. The percentage of lymphocytes in peripheral blood, which was decreased in 85.0% of patients (17/20) before treatment (mean, 15.52 ± 8.89%), returned to normal in 52.6% of patients (10/19) on the fifth day after treatment. Abnormally elevated CRP decreased significantly in 84.2% patients (16/19). No adverse drug reactions and no subsequent pulmonary infections were reported.

Nineteen patients (90.5%) were discharged at the time of the report, including two critical patients. There were no deaths among the 21 treated patients. The study authors concluded that TCZ is an effective treatment for patients with severe COVID-19 (Xu et al. (2020), supra).

An adaptive Phase ⅔, randomized, double-blind, placebo-controlled study assessing efficacy and safety of Sarilumab for hospitalized patients with COVID-19 is found at: https://www.clinicaltrials.gov/ct2/show/NCT04315298. Sarilumab is a human monoclonal antibody against the interleukin-6 receptor.

### SUMMARY OF THE INVENTION

The invention concerns a method of treating viral pneumonia in a patient comprising administering an effective amount of a combination of tocilizumab and remdesiver to the patient.

In one embodiment, the pneumonia is viral pneumonia.

In one embodiment, the pneumonia is coronavirus pneumonia.

In one embodiment, the pneumonia is COVID-19 pneumonia.

In one embodiment, the pneumonia is severe pneumonia.

In one embodiment, the pneumonia is severe COVID-19 pneumonia.

In one embodiment, the patient does not have an elevated IL-6 level.

In one embodiment, the patient has not been found to have an elevated IL-6 level by laboratory testing.

In one embodiment, the patient has alanine transaminase (ALT) or aspartate aminotransferase (AST) > 5 and < 10 upper limit of normal (ULN).

In one embodiment, the method treats acute respiratory distress (ARDS) in the patient.

### BRIEF DESCRIPTION OF THE DRAWINGS

FIG. 1 depicts the protocol for the clinical trial in Example 1.

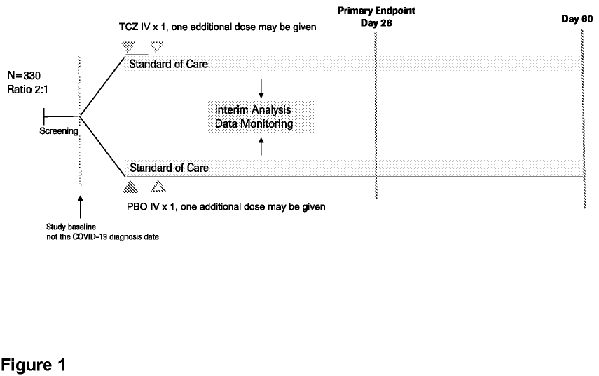

### DETAILED DESCRIPTION OF THE PREFERRED EMBODIMENTS

### I. Definitions

Abbreviations that may be used in this description

| | |
|---|---|
| Abbreviation | Definition |
| ARDS | acute respiratory distress syndrome |
| AUC | area under the curve |
| BAL | bronchoalveolar lavage |
| CAR | chimeric antigen receptor |
| Cₘₐₓ | maximum serum concentration observed |
| CMH | Cochran-Mantel-Haenszel |
| CoV | Coronaviruses |
| CRP | C-reactive protein |
| CRS | cytokine-release syndrome |
| CTCAE | Common Terminology Criteria for Adverse Events |
| ECMO | extracorporeal membrane oxygenation |
| FDA | Food and Drug Administration |
| GCA | giant cell arteritis |
| ICU | intensive care unit |
| IL-6 | interleukin 6 |
| IL-6R | interleukin-6 receptor |
| Iv | intravenous |
| MERS-CoV | Middle East respiratory syndrome |
| MOD | multiple organ dysfunction |
| MOF | multi organ failure |
| NEWS2 | National Early Warning Score 2 |
| PaO₂ | partial pressure of oxygen |
| PCR | polymerase chain reaction |
| pJIA | polyarticular juvenile idiopathic arthritis |
| PK | Pharmacokinetic |
| QW | once a week |
| Q2W | every 2 weeks |
| RA | rheumatoid arthritis |
| RT-PCR | real time polymerase chain reaction |
| SAE | serious adverse event |
| SAP | Statistical Analysis Plan |
| SARS-CoV | severe acute respiratory syndrome |
| sc | subcutaneous |
| sIL6-R | soluble interleukin-6 receptor |
| sJIA | systemic juvenile idiopathic arthritis |
| SOC | standard of care |
| SpO₂ | blood oxygen saturation |
| TAK | Takayasu arteritis |
| TB | Tuberculosis |
| TCZ | Tocilizumab |
| TTCI | time to clinical improvement |
| ULN | upper limit of normal |
| WHO | World Health Organization |

For the purposes herein “inflammation” refers to an immunological defense against infection, marked by increases in regional blood flow, immigration of white blood cells, and release of chemical toxins. Inflammation is one way the body uses to protect itself from infection. Clinical hallmarks of inflammation include redness, heat, swelling, pain, and loss of function of a body part. Systemically, inflammation may produce fevers, joint and muscle pains, organ dysfunction, and malaise.

“Pneumonia” refers to inflammation of one or both lungs, with dense areas of lung inflammation. The present invention concerns pneumonia due to viral infection. Symptoms of pneumonia may include fever, chills, cough with sputum production, chest pain, and shortness of breath. In one embodiment the pneumonia has been confirmed by chest X-ray or computed tomography (CT scan).

“Severe pneumonia” refers to pneumonia in which the heart, kidneys or circulatory system are at risk of failing, or if the lungs can no longer take in sufficient oxygen and develop acute respiratory distress syndrome (ARDS). A patient with severe pneumonia will typically be hospitalized and may be in an intensive care unit (ICU). Typically, the patient has severe dyspnea, respiratory distress, tachypnea (> 30 breaths/min), and hypoxia, optionally with fever. Cyanosis can occur in children. In this definition, the diagnosis is clinical, and radiologic imaging is used for excluding complications. In one embodiment, the patient with severe pneumonia has impaired lung function as determined by peripheral capillary oxygen saturation (SpO₂). In one embodiment, the patient with severe pneumonia has impaired lung function as determined by ratio of arterial oxygen partial pressure to fractional inspired oxygen (PaO2/FiO₂). In one embodiment, the patient with severe pneumonia has a SpO₂ ≤ 93%. In one embodiment, the patient with severe pneumonia has a PaO2/FiO₂ _{of} < 300 mmHg (optionally adjusted for high altitude areas based on PaO2/FiO₂ x [Atmospheric Pressure (mmHg)/760]). In one embodiment, the patient has respiratory distress (RR >30 breaths/minute). In one embodiment, the patient has > 50% lesions in pulmonary imaging.

“Critical pneumonia” refers to a severe pneumonia patient in whom respiratory failure, shock and/or organ has occurred. In one embodiment, the patient with critical pneumonia requires mechanical ventilation.

“Mild pneumonia” presents with symptoms of an upper respiratory tract viral infection, including mild fever, cough (dry), sore throat, nasal congestion, malaise, headache, muscle pain, or malaise. Signs and symptoms of a more serious disease, such as dyspnea, are not present.

In “Moderate Pneumonia”, respiratory symptoms such as cough and shortness of breath (or tachypnea in children) are present without signs of severe pneumonia. The patient with moderate pneumonia may be in a hospital, but not in an ICU or on a ventilator.

“Acute respiratory disease syndrome” or “ARDS” refers to a life-threatening lung condition that prevents enough oxygen from getting to the lungs and into the blood. In one embodiment, the diagnosis of ARDS is made based on the following criteria: acute onset, bilateral lung infiltrates on chest radiography of a non-cardiac origin, and a PaO/FiO ratio of < 300 mmHg. In one embodiment, the ARDS is “mild ARDS” characterized by PaO2/FiO₂ 200 to 300 mmHg. In one embodiment, the ARDS is “moderate ARDS” characterized by PaO2/FiO₂ 100 to 200 mmHg. In one embodiment, the ARDS is “severe ARDS” characterized by PaO2/FiO₂ < 100 mmHg.

“Viral pneumonia” refers to pneumonia caused by the entrance into a patient of one or more viruses. In one embodiment, the virus is a DNA virus. In one embodiment, the virus is an RNA virus. Examples of viruses causing viral pneumonia contemplated herein include, inter alia, those caused by: human immunodeficiency virus (HIV), hepatitis B virus, hepatitis C virus, influenza virus (including H1N1 or “swine flu” and H5N1 or “bird flu”), Zika virus, rotavirus, Rabies virus, West Nile virus, herpes virus, adenovirus, respiratory syncytial virus (RSV), norovirus, rotavirus, astrovirus, rhinovirus, human papillomavirus (HPV), polio virus, Dengue fever, Ebola virus, and coronavirus. In one embodiment, the viral pneumonia is caused by a coronavirus.

“Coronavirus” is a virus that infects humans and causes respiratory infection. Coronaviruses that can cause pneumonia in patients include, without limitation, the beta coronavirus causes Middle East Respiratory Syndrome (MERS), the beta coronavirus that causes severe acute respiratory syndrome (SARS), and the COVID-19 virus.

“COVID-19” refers to the virus that causes illness that is typically characterized by fever, cough, and shortness of breath and may progress to pneumonia and respiratory failure. COVID-19 was first identified in Wuhan China in December 2019. In one embodiment, the patient with COVID-19 is confirmed by positive polymerase chain reaction (PCR) test (e.g. real time PCT, RT-PCT test) of a specimen (e.g., respiratory, blood, urine, stool, other bodily fluid specimen) from the patient. In one embodiment, the COVID-19 nucleic acid sequence has been determined to be highly homologous to COVID-19. In one embodiment, the patient has COVID-19 specific antibodies (e.g. IgG and/or IgM antibodies), e.g. as determined by immunohistochemistry (IHC), enzyme-linked immunosorbent assay (ELISA), etc. Synonyms for COVID-19 include, without limitation, “novel coronavirus”, “2019 Novel Coronavirus” and “2019-nCoV”.

The term “patient” herein refers to a human patient.

An “intravenous” or “iv” dose, administration, or formulation of a drug is one which is administered via a vein, e.g. by infusion.

A “subcutaneous” or “sc” dose, administration, or formulation of a drug is one which is administered under the skin, e.g. via a pre-filled syringe, auto-injector, or other device.

A “weight-based dose” of a drug refers to a dose that is based on the weight of the patient. In a preferred embodiment, where the drug is tocilizumab, the weight-based dose is 8 mg/kg (optionally ≤ 800 mg dose).

A “fixed dose” of a drug refers to a dose that is administered without regard to the patient’s weight.

For the purposes herein, “clinical status” refers to a patient’s health condition. Examples include that the patient is improving or getting worse. In one embodiment, clinical status is based on an ordinal scale of clinical status. In one embodiment, clinical status is not based on whether or not the patient has a fever.

An “ordinal scale of clinical status” refers to a scale used to quantify outcomes which are non-dimensional. They include can include an outcome at a single point in time or can examine change which has occurred between two points in time. In one embodiment, the two points of time are “Day 1” (when first dose, e.g. 8 mg/kg, of the IL6 antagonist such as tocilizumab is administered) compared with “Day 28” (when the patient is evaluated) and, optionally, at “Day 60 (when the patient is further evaluated). Ordinal scales include various “categories” which each evaluate patent status or outcome. In one embodiment, the ordinal scale is a “7-category ordinal scale”.

In one embodiment, a “7-category ordinal scale” includes the following categories for evaluating the patient’s status:
1. Discharged from hospital (or “ready for discharge”, e.g. as evidenced by normal body temperature and respiratory rate, and stable oxygen saturation on ambient air or ≤ 2 L supplemental oxygen)
2. Non-ICU hospital ward (or “ready for hospital ward”) not requiring supplemental oxygen
3. Non-ICU hospital ward (or “ready for hospital ward”) requiring supplemental oxygen
4. ICU or non-ICU hospital ward, requiring non-invasive ventilation or high-flow oxygen
5. ICU, requiring intubation and mechanical ventilation
6. ICU, requiring ECMO or mechanical ventilation and additional organ support (e.g. vasopressors, renal replacement therapy)
7. Death.

For the purposes herein, “standard of care” or “SOC” refers to treatments or drugs commonly used to treat patients with pneumonia (e.g. viral pneumonia, such as COVID-19 pneumonia) including, inter alia, supportive care, administration of one or more anti-viral(s), and/or administration of one or more corticosteroid(s).

“Supportive care” includes, without limitation: respiratory support (e.g. oxygen therapy via face mask or nasal cannula, high-flow nasal oxygen therapy or non-invasive mechanical ventilation, invasive mechanical ventilation, via extracorporeal membrane oxygenation (ECMO), etc.); circulation support (e.g. fluid resuscitation, boost microcirculation, vasoactive drugs); renal replacement therapy; plasma therapy; blood purification therapy; Xuebijing Injection (e.g. 100 mL/day twice a day); microecological preparation (e.g. probiotics, prebiotics, and synbiotics); non-steroidal anti-inflammatory drugs (NSAIDs); herbal medicine; plasma (e.g. convalescent plasma) etc.

“Anti-viral” agents include, without limitation: alpha-interferon, lopinavir, ritonavir, lopinavir/ritonavir, remdesivir, ribavirin, hydroxychloroquine or chloroquine (with or without azithromycin), umifenovir, etc.

“Corticosteroid” refers to any one of several synthetic or naturally occurring substances with the general chemical structure of steroids that mimic or augment the effects of the naturally occurring corticosteroids. Examples of synthetic corticosteroids include prednisone, prednisolone (including methylprednisolone, such as methylprednisolone sodium succinate), dexamethasone or dexamethasone triamcinolone, hydrocortisone, and betamethasone. In one embodiment, the corticosteroid is selected from prednisone, methylprednisolone, hydrocortisone, and dexamethasone. In one embodiment, the corticosteroid is methylprednisolone. In one embodiment, the corticosteroid is “low-dose” glucocorticoid (e.g. ≤ 1-2 mg/kg/day methylprednisolone, e.g. for 3-5 days).

Herein “human interleukin 6” (abbreviated as “IL-6”) is a cytokine also known as B cell-stimulating factor 2 (BSF-2), or interferon beta-2 (IFNB2), hybridoma growth factor, and CTL differentiation factor. IL-6 was discovered as a differentiation factor contributing to activation of B cells (Hirano et al., Nature 324: 73-76 (1986)), and was later found to be a multifunction cytokine which influences the functioning of a variety of different cell types (Akira et al., Adv. in Immunology 54: 1-78 (1993)). Naturally occurring human IL-6 variants are known and included in this definition. Human IL-6 amino acid sequence information has been disclosed, see for example, www.uniprot.org/uniprot/P05231.

An “IL6 antagonist” refers to agent that inhibits or blocks IL6 biological activity via binding to human IL6 or human IL6 receptor. In one embodiment, the IL6 antagonist is an antibody. In one embodiment, the IL6 antagonist is an antibody that binds IL6 receptor. Antibodies that bind IL-6 receptor include tocilizumab (including intravenous, iv, and subcutaneous sc formulations thereof) (Chugai, Roche, Genentech), satralizumab (Chugai, Roche, Genentech), sarilumab (Sanofi, Regeneron), NI-1201 (Novimmune and Tiziana), and vobarilizumab (Ablynx). In one embodiment, the IL6 antagonist is a monoclonal antibody that binds IL6. Antibodies that bind IL-6 include sirukumab (Centecor, Janssen), olokizumab (UCB), clazakizumab (BMS and Alder), siltuximab (Janssen), EBI-031 (Eleven Biotherapeutics and Roche). In one embodiment, the IL6 antagonist is olamkicept.

For the purposes herein “human interleukin 6 receptor” (abbreviated as “IL-6R”) refers to the receptor which binds IL-6, including both membrane-bound IL-6R (mIL-6R) and soluble IL-6R (sIL-6R). IL-6R can combine with interleukin 6 signal transducer glycoprotein 130 to form an active receptor complex. Alternatively spliced transcript variants encoding distinct isoforms of IL-6 have been reported and are included in this definition. The amino acid sequence structure of human IL-6R and its extracellular domain have been described; see, for example, Yamasaki et al., Science, 241: 825 (1988).

A “neutralizing” anti-IL-6R antibody herein is one which binds to IL-6R and is able to inhibit, to a measurable extent, the ability of IL-6 to bind to and/or active IL-6R. Tocilizumab is an example of a neutralizing anti-IL-6R antibody.

“Tocilizumab” or “TCZ” is a recombinant humanized monoclonal antibody that binds to human interleukin-6 receptor (IL-6R). It is an IgG1κ (gamma 1, kappa) antibody with a two heavy chains and two light chains forming two antigen-binding sites. In a preferred embodiment, the light chain and heavy chain amino acid sequences of Tocilizumab comprise SEQ ID NOs. 1 and 2, respectively.

A “native sequence” protein herein refers to a protein comprising the amino acid sequence of a protein found in nature, including naturally occurring variants of the protein. The term as used herein includes the protein as isolated from a natural source thereof or as recombinantly produced.

The term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity.

“Antibody fragments” herein comprise a portion of an intact antibody which retains the ability to bind antigen. Examples of antibody fragments include Fab, Fab′, F(ab′)₂, and Fv fragments; diabodies; linear antibodies; single-chain antibody molecules; and multispecific antibodies formed from antibody fragments.

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example. Specific examples of monoclonal antibodies herein include chimeric antibodies, humanized antibodies, and human antibodies, including antigen-binding fragments thereof.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences (US Pat No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence, except for FR substitution(s) as noted above. The humanized antibody optionally also will comprise at least a portion of an immunoglobulin constant region, typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). Humanized antibodies herein specifically include “reshaped” IL-6R antibodies as described in U.S. Pat. No. 5,795,965, expressly incorporated herein by reference.

A “human antibody” herein is one comprising an amino acid sequence structure that corresponds with the amino acid sequence structure of an antibody obtainable from a human B-cell, and includes antigen-binding fragments of human antibodies. Such antibodies can be identified or made by a variety of techniques, including, but not limited to: production by transgenic animals (e.g., mice) that are capable, upon immunization, of producing human antibodies in the absence of endogenous immunoglobulin production (see, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and U.S. Pat. Nos. 5,591,669, 5,589,369 and 5,545,807)); selection from phage display libraries expressing human antibodies or human antibody fragments (see, for example, McCafferty et al., Nature 348:552-553 (1990); Johnson et al., Current Opinion in Structural Biology 3:564-571 (1993); Clackson et al., Nature, 352:624-628 (1991); Marks et al., J. Mol. Biol. 222:581-597 (1991); Griffith et al., EMBO J. 12:725-734 (1993);U.S. Pat. Nos. 5,565,332 and 5,573,905); generation via in vitro activated B cells (see U.S. Pats. 5,567,610 and 5,229,275); and isolation from human antibody producing hybridomas.

A “multispecific antibody” herein is an antibody having binding specificities for at least two different epitopes. Exemplary multispecific antibodies may bind to two different epitopes of IL-6R. Alternatively, an anti-IL-6R binding arm may be combined with an arm that binds to a triggering molecule on a leukocyte such as a T-cell receptor molecule (e.g. CD2 or CD3), or Fc receptors for IgG (FcγR), such as FcγRI (CD64), FcγRII (CD32) and FcγRIII (CD16) so as to focus cellular defense mechanisms to the receptor. Multispecific antibodies can be prepared as full-length antibodies or antibody fragments (e.g. F(ab′)₂ bispecific antibodies). Engineered antibodies with three or more (preferably four) functional antigen binding sites are also contemplated (see, e.g., U.S. Appln. No. US 2002/0004587 A1, Miller et al.).

Antibodies herein include “amino acid sequence variants” with altered antigen-binding or biological activity. Examples of such amino acid alterations include antibodies with enhanced affinity for antigen (e.g. affinity matured antibodies), and antibodies with altered Fc region, if present, e.g. with altered (increased or diminished) antibody dependent cellular cytotoxicity (ADCC) and/or complement dependent cytotoxicity (CDC) (see, for example, WO 00/42072, Presta, L. and WO 99/51642, Iduosogie et al.); and/or increased or diminished serum half-life (see, for example, WO00/42072, Presta, L.).

The antibody herein may be conjugated with a “heterologous molecule” for example to increase half-life or stability or otherwise improve the antibody. For example, the antibody may be linked to one of a variety of non-proteinaceous polymers, e.g., polyethylene glycol (PEG), polypropylene glycol, polyoxyalkylenes, or copolymers of polyethylene glycol and polypropylene glycol. Antibody fragments, such as Fab′, linked to one or more PEG molecules are an exemplary embodiment of the invention.

The antibody herein may be a “glycosylation variant” such that any carbohydrate attached to the Fc region, if present, is altered. For example, antibodies with a mature carbohydrate structure that lacks fucose attached to an Fc region of the antibody are described in U.S. Pat. Appl. No. US 2003/0157108 (Presta, L.). See also US 2004/0093621 (Kyowa Hakko Kogyo Co., Ltd). Antibodies with a bisecting N-acetylglucosamine (GlcNAc) in the carbohydrate attached to an Fc region of the antibody are referenced in WO 2003/011878, Jean-Mairet et al. and U.S. Pat. No. 6,602,684, Umana et al. Antibodies with at least one galactose residue in the oligosaccharide attached to an Fc region of the antibody are reported in WO 1997/30087, Patel et al. See, also, WO 1998/58964 (Raju, S.) and WO 1999/22764 (Raju, S.) concerning antibodies with altered carbohydrate attached to the Fc region thereof. See also US 2005/0123546 (Umana et al.) describing antibodies with modified glycosylation.

The term “hypervariable region” when used herein refers to the amino acid residues of an antibody that are responsible for antigen binding. The hypervariable region comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins oƒ Immunological Interest, 5th Ed. Public Health Service, National Institutes of Health, Bethesda, MD. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J.Mol. Biol. 196:901-917 (1987)). “Framework” or “FR” residues are those variable domain residues other than the hypervariable region residues as herein defined. The hypervariable regions of Tocilizumab comprise:
L1 - Arg Ala Ser Gln Asp I1e Ser Tyr Leu Asn (SEQ ID NO: 3);
L2 - Tyr Thr Ser Arg Leu His Ser (SEQ ID NO: 4);
L3 - Gln Gly Asn Thr Leu Pro Tyr Thr (SEQ ID NO:5);
H1 - Ser Asp His Ala Trp Ser (SEQ ID NO:6);
H2 - Tyr Ile Ser Tyr Ser Gly Ile Thr Tyr Asn Pro Ser Leu Lys Ser (SEQ ID NO:7); and
H3 - Ser Leu Ala Arg Thr Ala Met Asp Tyr (SEQ ID NO:8).

In one embodiment herein, the IL-6R antibody comprises the hypervariable regions of Tocilizumab.

A “full length antibody” is one which comprises an antigen-binding variable region as well as a light chain constant domain (CL) and heavy chain constant domains, CH1, CH2 and CH3. The constant domains may be native sequence constant domains (e.g. human native sequence constant domains) or amino acid sequence variants thereof. Preferably, the full length antibody has one or more effector functions. Tocilizumab is an example of a full-length antibody.

A “naked antibody” is an antibody (as herein defined) that is not conjugated to a heterologous molecule, such as a cytotoxic moiety, polymer, or radiolabel.

Antibody “effector functions” refer to those biological activities attributable to the Fc region (a native sequence Fc region or amino acid sequence variant Fc region) of an antibody. Examples of antibody effector functions include C1q binding, complement dependent cytotoxicity (CDC), Fc receptor binding, antibody-dependent cell-mediated cytotoxicity (ADCC), etc.

Depending on the amino acid sequence of the constant domain of their heavy chains, full length antibodies can be assigned to different “classes”. There are five major classes of full length antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known.

The term “recombinant antibody”, as used herein, refers to an antibody (e.g. a chimeric, humanized, or human antibody or antigen-binding fragment thereof) that is expressed by a recombinant host cell comprising nucleic acid encoding the antibody. Examples of “host cells” for producing recombinant antibodies include: (1) mammalian cells, for example, Chinese Hamster Ovary (CHO), COS, myeloma cells (including Y0 and NS0 cells), baby hamster kidney (BHK), Hela and Vero cells; (2) insect cells, for example, sf9, sf21 and Tn5; (3) plant cells, for example plants belonging to the genus Nicotiana (e.g. Nicotiana tabacum); (4) yeast cells, for example, those belonging to the genus Saccharomyces (e.g. Saccharomyces cerevisiae) or the genus Aspergillus (e.g. Aspergillus niger); (5) bacterial cells, for example Escherichia coli cells or Bacillus subtilis cells, etc.

As used herein, “specifically binding” or “binds specifically to” refers to an antibody selectively or preferentially binding to IL-6R antigen. Preferably the binding affinity for antigen is of Kd value of 10⁻⁹ mol/l or lower (e.g. 10⁻¹⁰ mol/l), preferably with a Kd value of 10⁻¹⁰ mol/l or lower (e.g. 10⁻¹² mol/l). The binding affinity is determined with a standard binding assay, such as surface plasmon resonance technique (BIACORE®).

Examples of “non-steroidal anti-inflammatory drugs” or “NSAIDs” include aspirin, acetylsalicylic acid, ibuprofen, flurbiprofen, naproxen, indomethacin, sulindac, tolmetin, phenylbutazone, diclofenac, ketoprofen, benorylate, mefenamic acid, methotrexate, fenbufen, azapropazone; COX-2 inhibitors such as celecoxib (CELEBREX®; 4-(5-(4-methylphenyl)-3-(trifluoromethyl)-1H-pyrazol-1-yl) benzenesulfonamide, valdecoxib (BEXTRA®), meloxicam (MOBIC®), GR 253035 (Glaxo Wellcome); and MK966 (Merck Sharp & Dohme), including salts and derivatives thereof, etc. Specific embodiments include: aspirin, naproxen, ibuprofen, indomethacin, and tolmetin.

The expression “effective amount” refers to an amount of the IL6 antagonist (e.g. IL6 receptor antibody such as tocilizumab) that is effective for treating pneumonia (e.g. viral pneumonia, including COVID-19 pneumonia) and/or for treating acute respiratory distress syndrome (ARDS).

The term “pharmaceutical formulation” refers to a preparation which is in such form as to permit the biological activity of the active ingredient or ingredients to be effective, and which contains no additional components which are unacceptably toxic to a subject to which the formulation would be administered. Such formulations are sterile. In one embodiment, the formulation is for intravenous (iv) administration. In another embodiment, the formulation is for subcutaneous (sc) administration.

A “sterile” formulation is aseptic or free from all living microorganisms and their spores.

A “liquid formulation” or “aqueous formulation” according to the invention denotes a formulation which is liquid at a temperature of at least about 2 to about 8° C.

The term “lyophilized formulation” denotes a formulation which is dried by freezing the formulation and subsequently subliming the ice from the frozen content by any freeze-drying methods known in the art, for example commercially available freeze-drying devices. Such formulations can be reconstituted in a suitable diluent, such as water, sterile water for injection, saline solution etc., to form a reconstituted liquid formulation suitable for administration to a subject.

A “package insert” is used to refer to instructions customarily included in commercial packages of therapeutic products, that contain information about the indications, usage, dosage, administration, contraindications, other therapeutic products to be combined with the packaged product, and/or warnings concerning the use of such therapeutic products, etc.

An “elevated” level of a biomarker refers to an amount of that biomarker in the patient that is above the upper limit of normal (ULN).

An “elevated IL6 level” is ≥ 15 pg/mL, or ≥ 10 pg/mL or > 7 pg/mL, e.g. as measured by enzyme linked immunosorbent assay (ELISA) of a blood sample from the patient. In one embodiment, “normal” IL6 level is considered to be 7 pg/mL.

The patient who has “not been found to have elevated IL-6 levels by laboratory testing” has been treated according to the methods herein without regard to his or her IL-6 level. In one embodiment, such patient does not have an elevated IL6 level.

“Remdesivir” is an antiviral medication, a nucleotide analog, specifically an adenosine analogue, which inserts into viral RNA chains, causing their premature termination. Its molecular formula is C₂₇H₃₅N₆O₈P and IUPAC Name is 2-ethylbutyl (2,S)-2-[[[(2R,3S,4R,5R)-5-(4-aminopyrrolo[2,1-f][1,2,4]triazin-7-yl)-5-cyano-3,4-dihydroxyoxolan-2-yl]methoxy-phenoxyphosphoryl]amino]propanoate. Remdesivir’s laboratory name is GS-5734 and its CAS number is 1809249-37-3. It is described in U.S. Pat. No. 9,724,360 and is manufactured by Gilead Sciences.

### II. Production of IL6 Antagonists

IL6 antagonists contemplated herein include antagonists that bind to IL6 or IL6 receptor.

In one embodiment, the IL6 antagonist is an antibody.

In one embodiment, the IL6 antagonist is an antibody that binds IL6 receptor.

In one embodiment, the IL6 antagonist is an antibody that binds to both membrane-bound IL6 receptor and soluble IL6 receptor.

In one embodiment, the IL6 antagonist blocks the IL-6/IL-6 receptor complex as well as depleting circulating levels of IL-6 in the blood.

Antibodies that bind IL-6 receptor include tocilizumab (including intravenous, iv, and subcutaneous sc formulations thereof) (Chugai, Roche, Genentech), satralizumab (Chugai, Roche, Genentech), sarilumab (Sanofi, Regeneron), NI-1201 or TZLS-501 (Novimmune and Tiziana), and vobarilizumab (Ablynx).

In one embodiment, the IL6 antagonist is tocilizumab.

Tocilizumab, also named Myeloma Receptor Antibody (MRA), is a recombinant humanized monoclonal antibody that selectively binds to human interleukin-6 receptor (IL-6R). It is an IgG1κ (gamma 1, kappa) antibody with a typical H₂L₂ structure. The tocilizumab molecule is composed of two heterodimers. Each of the heterodimers is composed of a heavy (H) and a light (L) polypeptide chain. The four polypeptide chains are linked intra- and inter-molecularly by disulfide linkages. The molecular formula and theoretical molecular weight of the tocilizumab antibody are as follows:
Molecular formula: C₆₄₂₈H₉₉₇₆N₁₇₂₀O₂₀₁₈S₄₂ (polypeptide moiety only)
Molecular weight: 144,985 Da (polypeptide moiety only).

The amino acid sequence of the light chain deduced from complimentary deoxyribonucleic acid (cDNA) sequences and confirmed by liquid chromatography mass-spectrometry (LC-MS) peptide mapping is in SEQ ID Nos. 1 and 2. The five light chain cysteine residues of each heterodimer are involved in two intrachain disulfide linkages and one interchain disulfide linkage:
Intrachain linkages: Cys_{L23}-Cys_{L88} and Cys_{L134}-Cys_{L194}
Linkage between heavy and light chain: Cys_{L214} and Cys_{H222}

Assignments of the disulfide linkages are based on sequence homology to other IgG1 antibodies and were confirmed by liquid chromatography mass-spectrometry (LC-MS) peptide mapping performed using material from the fourth generation (G4) process. Cys_{Lx} and Cys_{Hx} denote cysteine residues at position x of the light and heavy chains, respectively.

SEQ ID NO. 1

Amino Acid Sequence of the L Chain of the Tocilizumab Molecule

| | | | | | | |
|---|---|---|---|---|---|---|
| 1 | DIQMTQSPSS | LSASVGDRVT | ITCRASQDIS | SYLNWYQQKP | GKAPKLLIYY | 50 |
| 51 | TSRLHSGVPS | RFSGSGSGTD | FTFTISSLQP | EDIATYYCQQ | GNTLPYTFGQ | 100 |
| 101 | GTKVEIKRTV | AAPSVFIFPP | SDEQLKSGTA | SVVCLLNNFY | PREAKVQWKV | 150 |
| 151 | DNALQSGNSQ | ESVTEQDSKD | STYSLSSTLT | LSKADYEKHK | VYACEVTHQG | 200 |
| 201 | LSSPVTKSFN | RGEC | | | | 214 |

Note: The entire sequence has been determined by LC-MS peptide mapping.

The amino acid sequence of the heavy chain deduced from complimentary deoxyribonucleic acid (cDNA) sequences and confirmed by amino acid sequencing is in SEQ ID NO. 2. The eleven heavy chain cysteine residues of each heterodimer are involved in four intrachain disulfide linkages, two interchain disulfide linkages between the two heavy chains and the third interchain disulfide linkage between the heavy chain and the light chain of each of the heterodimers:
Intrachain linkages: Cys_{H22}-Cys_{H96}, Cys_{H146}-CyS_{H202}, Cys_{H263}-Cys_{H323} and Cys_{H369}-Cys_{H427}
Linkages between the two heavy chains: Cys_{H228}-Cys_{H228} and Cys_{H231}-Cys_{H231}
Linkage between heavy and light chain: Cys_{L214}-Cys_{H222}

Assignments of the disulfide linkages are based on sequence homology to other IgG1 antibodies and were confirmed by LC-MS peptide mapping performed using material from the G4 process.

SEQ ID NO. 2

Amino Acid Sequence of the H Chain of the Tocilizumab Molecule

| | | | | | | |
|---|---|---|---|---|---|---|
| 1 | pEVQLQESGPG | LVRPSQTLSL | TCTVSGYSIT | SDHAWSWVRQ | PPGRGLEWIG | 50 |
| 51 | YISYSGITTY | NPSLKSRVTM | LRDTSKNQFS | LRLSSVTAAD | TAVYYCARSL | 100 |
| 101 | ARTTAMDYWG | QGSLVTVSSA | STKGPSVFPL | APSSKSTSGG | TAALGCLVKD | 150 |
| 151 | YFPEPVTVSW | NSGALTSGVH | TFPAVLQSSG | LYSLSSVVTV | PSSSLGTQTY | 200 |
| 201 | ICNVNHKPSN | TKVDKKVEPK | SCDKTHTCPP | CPAPELLGGP | SVFLFPPKPK | 250 |
| 251 | DTLMISRTPE | VTCVVVDVSH | EDPEVKFNWY | VDGVEVHNAK | TKPREEQYNS | 300 |
| 301 | TYRWSVLTV | LHQDWLNGKE | YKCKVSNKAL | PAPIEKTISK | AKGQPREPQV | 350 |
| 351 | YTLPPSRDEL | TKNQVSLTCL | VKGFYPSDIA | VEWESNGQPE | NNYKTTPPVL | 400 |
| 401 | DSDGSFFLYS | KLTVDKSRWQ | QGNVFSCSVM | HEALHNHYTQ | KSLSLSPG | 448 |

Note: The entire sequence has been determined by LC-MS peptide mapping. The N-terminus of the heavy chain has been determined to be predominantly a pyroglutamic acid residue (pE).

In one embodiment, the IL6 antagonist is satralizumab. Satralizumab (also called SA237) is a humanized monoclonal antibody that binds IL6 receptor. See U.S. Pat. No. US 8,562,991.

In one embodiment, the IL6 antagonist is the human antibody that binds the IL6 receptor called TZLS-501 (Tiziana) or NI-1201 (Novimmune).

In one embodiment, the IL6 antagonist is a monoclonal antibody that binds IL6.

Antibodies that bind IL-6 include sirukumab (Centecor, Janssen), olokizumab (UCB), clazakizumab (BMS and Alder), siltuximab (Janssen), EBI-031 (Eleven Biotherapeutics and Roche).

In one embodiment, the IL6 antagonist is olamkicept. Olamkicept is a recombinant protein that fuses the extracellular domain of the signal transducing subunit of the IL-6 receptor, IL-6Rβ (glycoprotein 130, gp130), to a human IgG Fc fragment. The full construct is a dimer of covalently linked identical peptide chains. Mechanistically olamkicept acts as an inhibitor of the IL-6 signaling pathway. Olamkicept inhibits trans-signaling by the soluble IL-6 receptor (sIL-6R).

In a preferred embodiment, the methods and articles of manufacture of the present invention use, or incorporate, an antibody that binds to human IL-6R. IL-6R antigen to be used for production of, or screening for, antibodies may be, e.g., a soluble form of IL-6R or a portion thereof (e.g. the extracellular domain), containing the desired epitope. Alternatively, or additionally, cells expressing IL-6R at their cell surface can be used to generate, or screen for, antibodies. Other forms of IL-6R useful for generating antibodies will be apparent to those skilled in the art.

In one embodiment, the antibody is an antibody fragment, various such fragments being disclosed above.

In another embodiment, the antibody is an intact or full-length antibody. Depending on the amino acid sequence of the constant domain of their heavy chains, intact antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy chain constant domains that correspond to the different classes of antibodies are called α, δ, ε, γ, and µ, respectively. The subunit structures and three-dimensional configurations of different classes of immunoglobulins are well known. In a preferred embodiment, the anti-IL-6R antibody is an IgG1 or IgM antibody.

Techniques for generating antibodies are known and examples provided above in the definitions section of this document. In a preferred embodiment, the antibody is a chimeric, humanized, or human antibody or antigen-binding fragment thereof. Preferably the antibody is a humanized full-length antibody.

Various techniques are available for determining binding of the antibody to the IL-6R. One such assay is an enzyme linked immunosorbent assay (ELISA) for confirming an ability to bind to human IL-6R. See, for example, U.S. Pat. No. 5,795,965. According to this assay, plates coated with IL-6R (e.g. recombinant sIL-6R) are incubated with a sample comprising the anti-IL-6R antibody and binding of the antibody to the sIL-6R is determined.

Preferably, the anti-IL-6R antibody is neutralizes IL-6 activity, e.g. by inhibiting binding of IL-6 to IL-6R. An exemplary method for evaluating such inhibition is disclosed in U.S. Pat. Nos. 5,670,373, and 5,795,965, for example. According to this method, the ability of the antibody to compete with IL-6 to IL-6R is evaluated. For example, a plate is coated with IL-6R (e.g. recombinant sIL-6R), a sample comprising the anti-IL-6R antibody with labeled IL-6 is added, and the ability of the antibody to block binding of the labeled IL-6 to the IL-6R is measured. See, U.S. Pat. No. 5,795,965. Alternatively, or additionally, identification of binding of IL-6 to membrane-bound IL-6R is carried out according to the method of Taga et al. J. Exp. Med., 166: 967 (1987). An assay for confirming neutralizing activity using the IL-6-dependent human T-cell leukemia line KT3 is also available, see, U.S. Pat. No. 5,670,373, and Shimizu et al. Blood 72: 1826 (1988).

Non-limiting examples of anti-IL-6R antibodies herein include PM-1 antibody (Hirata et al., J. Immunol. 143:2900-2906 (1989), AUK12-20, AUK64-7, and AUK146-15 antibody (U.S. Pat. No. 5,795,965), as well as humanized variants thereof, including, for example, tocilizumab. See, U.S. Pat. No. 5,795,965. Preferred examples of the reshaped human antibodies used in the present invention include humanized or reshaped anti-interleukin (IL-6) receptor antibodies (hPM-1 or MRA) (see US Patent No. 5,795,965).

The antibody herein is preferably recombinantly produced in a host cell transformed with nucleic acid sequences encoding its heavy and light chains (e.g. where the host cell has been transformed by one or more vectors with the nucleic acid therein). The preferred host cell is a mammalian cell, most preferably a Chinese Hamster Ovary (CHO) cells.

### III. Pharmaceutical Formulations

Therapeutic formulations of the antibodies used in accordance with the present invention are prepared for storage by mixing an antibody having the desired degree of purity with optional pharmaceutically acceptable carriers, excipients or stabilizers (Remington’s Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The formulation herein may also contain more than one active compound as necessary, preferably those with complementary activities that do not adversely affect each other. The type and effective amounts of such medicaments depend, for example, on the amount of antibody present in the formulation, and clinical parameters of the subjects. Exemplary such medicaments are discussed below.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington’s Pharmaceutical Sciences 16th edition, Osol, A. Ed. (1980).

Sustained-release preparations may be prepared. Suitable examples of sustained-release preparations include semi-permeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(-)-3-hydroxybutyric acid.

The formulations to be used for in vivo administration must be sterile. This is readily accomplished by filtration through sterile filtration membranes.

In one embodiment, the formulation is suitable for intravenous (iv) infusion, for example, the tocilizumab iv formulation as disclosed in U.S. Pat. Nos. 8,840,884 and 9,051,384. In one embodiment, a tocilizumab iv formulation is a sterile, clear, colorless to pale yellow, preservative-free solution for further dilution prior to intravenous infusion with a pH of approximately 6.5. In one embodiment, a tocilizumab iv formulation is supplied in a single-dose vial, formulated with a disodium phosphate dodecahydrate/sodium dihydrogen phosphate dihydrate buffered solution, and is available at a concentration of 20 mg/mL containing 80 mg/4 mL, 200 mg/10 mL, or 400 mg/20 mL of tocilizumab. In one embodiment, each mL of tocilizumab iv solution contains polysorbate 80 (0.5 mg), sucrose (50 mg), and Water for Injection, USP.

In one embodiment, the formulation is suitable for subcutaneous (sc) administration, for example, the tocilizumab sc formulation as in U.S. Pat. 8,568,720. In one embodiment, a tocilizumab sc formulation is a sterile, clear, colorless to slightly yellowish, preservative-free, histidine buffered solution for subcutaneous use with a pH of approximately 6.0. In one embodiment, a tocilizumab sc formulation is supplied in a ready-to-use, single-dose 0.9 mL prefilled syringe (PFS) with a needle safety device, or a ready-to-use, single-dose 0.9 mL autoinjector. In one embodiment tocilizumab sc formulation delivers 162 mg tocilizumab, L-arginine hydrochloride (19 mg), L-histidine (1.52 mg), L-histidine hydrochloride monohydrate (1.74 mg), L-methionine (4.03 mg), polysorbate 80 (0.18 mg), and Water for Injection.

Preferably the formulation is isotonic.

### IV. Therapeutic Uses of Anti-IL-6 Antagonists

The invention provides a method of treating pneumonia in a patient comprising administering a (first) weight-based intravenous dose of tocilizumab to the patient, wherein the weight-based dose is 8 mg/kg of tocilizumab (e.g. wherein ≤ 800 mg of tocilizumab is administered to the patient).

In one embodiment, the pneumonia is severe pneumonia.

In one embodiment, the pneumonia is critical pneumonia.

In one embodiment, the pneumonia is moderate pneumonia.

In one embodiment, the pneumonia is moderate-severe pneumonia.

In one embodiment, the pneumonia is viral pneumonia.

In one embodiment, the viral pneumonia is coronavirus pneumonia.

In one embodiment, the pneumonia is COVID-19 pneumonia, Middle East respiratory syndrome (MERS-CoV) pneumonia, or severe acute respiratory syndrome (SARS-CoV) pneumonia.

In one embodiment, the viral pneumonia is COVID-19 pneumonia.

In one embodiment, the viral pneumonia is severe COVID-19 pneumonia.

In one embodiment, the viral pneumonia is critical COVID-19 pneumonia.

In one embodiment, the viral pneumonia is moderate COVID-19 pneumonia.

In one embodiment, the viral pneumonia is moderate-severe COVID-19 pneumonia.

In one embodiment, the method further comprises administering a single (second) weight-based intravenous dose of tocilizumab to the patient 8-12 hours (or 8-24 hours) after the first dose, wherein the second weight-based dose is 8 mg/kg (e.g. wherein ≤ 800 mg of tocilizumab is administered to the patient with the second dose).

In one embodiment, the method further comprises administering a single (second) weight-based intravenous dose of tocilizumab to the patient 8-11 hours after the first dose, wherein the second weight-based dose is 8 mg/kg (e.g. wherein ≤ 800 mg of tocilizumab is administered to the patient with the second dose).

In one embodiment, only a single weight-based dose, 8 mg/kg (≤800 mg) is administered to the patient.

In one embodiment, only two weight-based doses, each being 8 mg/kg (each ≤800 mg), are administered to the patient.

In one embodiment, the second dose is administered after the patient experiences no improvement or worsening of clinical status after the first dose.

In one embodiment, the second dose is administered after the patient experiences no improvement or ≥ one-category worsening on an ordinal scale of clinical status following the first dose.

In one embodiment, the second dose is administered after the patient experiences ≥ one-category worsening on an ordinal scale of clinical status following the first dose.

In one embodiment, the ordinal scale is a 7-category ordinal scale.

The invention provides methods of treating pneumonia (e.g. viral pneumonia, coronavirus pneumonia, or COVID-19 pneumonia) with an anti-IL6 antagonist (e.g. an anti-IL6 receptor antibody such as tocilizumab, sarliumab, satralizumab, and/or TZLS-501), which achieves a greater improvement in clinical outcome than standard of care (SOC).

Methods for confirming the improvement in clinical outcome compared with SOC, include, without limitation, any one or more of the following:
1. clinical outcome measured on an ordinal scale of clinical status (e.g. at Day 28 and/or Day 60);
2. clinical outcome measured on a 7-category ordinal scale of clinical status (e.g. at Day 28 and/or Day 60);
3. clinical outcome comprising time to improvement of at least 2 categories relative to baseline on a 7-category ordinal scale of clinical status (e.g. at Day 28 and/or Day 60);
4. clinical outcome comprising time to clinical improvement (TTCI) defined as a National Early Warning Score 2 (NEWS2) of ≤ 2 maintained for 24 hours;
5. incidence of mechanical ventilation (e.g. at Day 28 and/or Day 60);
6. ventilator-free days (e.g. to Day 28);
7. organ failure-free days (e.g. to Day 28 and/or Day 60);
8. incidence of intensive care unit (ICU) stay (e.g. to Day 28 and/or Day 60);
9. duration of ICU stay (e.g. to Day 28 and/or Day 60);
10. time to clinical failure, e.g. defined as the time to death, mechanical ventilation, ICU admission, or withdrawal, whichever occurs first;
11. mortality rate (e.g. at Days 7, 14, 21, 28, and 60 following treatment on Day 1).
12. time to hospital discharge;
13. time to ready for discharge (e.g. as evidenced by normal body temperature and respiratory rate, and stable oxygen saturation on ambient air or ≤ 2 L supplemental oxygen);
14. duration of supplemental oxygen;
15. incidence of vasopressor use;
16. duration of vasopressor use;
17. incidence of extracorporeal membrane oxygenation (ECMO);
18. duration of ECMO;

In one embodiment, the method of treatment with the IL6 antagonist is associated with acceptable safety outcome compared with standard of care (SOC). Exemplary safety outcomes include any one or more of:
1. incidence and severity of adverse events;
2. severity of adverse events determined according to National Cancer Institute Common Terminology Criteria for Adverse Events (NCI CTCAE) v5.0;
3. COVID-19 (SARS-CoV-2) viral load over time;
4. time to reverse-transcriptase polymerase chain reaction (RT-PCR) virus negativity;
5. post-treatment infection; and
6. change from baseline in targeted clinical laboratory test results.

Herein, SOC for pneumonia, in particular viral pneumonia (such as COVID-19 pneumonia) includes any one or more of (e.g. one, two, or three of):
1. supportive care;
2. one or more anti-viral agent(s);
3. one or more corticosteroid(s), e.g. low dose corticosteroid(s).

In one embodiment, the SOC comprises supportive care. Example of supportive care, include, without limitation:
1. oxygen therapy (e.g. via face mask or nasal cannula; high-flow nasal oxygen therapy or non-invasive mechanical ventilation; invasive mechanical ventilation; lung expansion via extracorporeal membrane oxygenation (ECMO), etc.);
2. circulation support (e.g. fluid resuscitation, boost microcirculation, and/or vasoactive drugs);
3. renal replacement therapy;
4. plasma therapy;
5. blood purification therapy;
6. Xuebijing Injection (e.g. 100 mL/day twice a day); and
7. microecological agents (e.g. probiotics, prebiotics, and synbiotics), etc.

In one embodiment, the SOC includes treatment with one or more anti-viral agents (preferably only one or two) anti-viral agent(s). Exemplary anti-viral treatments include, without limitation:
1. alpha-interferon (e.g. via nebulization; e.g. about 5 million units or equivalent per time for adult, add 2 mL of sterile water for injection; e.g. via aerosol inhalation twice per day);
2. lopinavir/ritonavir (e.g. 200 mg/50 mg per capsule, 2 capsules each time, twice per day for adults, e.g. ≤10 days);
3. ribavirin (e.g. combined with alpha-interferon or lopinavir/ritonavir, e.g. 500 mg for adults per time, 2-3 times per day intravenously, e.g. ≤10 days);
4. Chloroquine phosphate or hydroxychloroquine (e.g. for adults from 18 to 65 years of age; e.g. if the body weight is greater than 50 kg, 500 mg per time, twice per day for 7 days; if the body weight is less than 50 kg, 500 mg per time, twice per day for day 1 and day 2; 500 mg per time, once per day for day 3 to 7), optionally together with azithromycin; and
5. Umifenovir (e.g. 200 mg for adults, e.g. three times per day, e.g. ≤10 days).

In one embodiment, the SOC includes treatment with corticosteroid(s), e.g.
1. wherein the patient has progressive deterioration of oxygenation, rapid X-ray progression, and/or excessive inflammatory response;
2. prednisone, prednisolone, methylprednisolone, methylprednisolone sodium succinate, dexamethasone, dexamethasone triamcinolone, hydrocortisone, and/or betamethasone;
3. prednisone, methylprednisolone, hydrocortisone, or dexamethasone.
4. methylprednisolone;
5. “low dose” corticosteroid;
6. corticosteroid administered ≤ 1-2 mg/kg/day;
7. methylprednisolone ≤ 1-2 mg/kg/day;
8. methylprednisolone ≤ 1-2 mg/kg/day for 3-5 days.

The invention also concerns a method of treating pneumonia (including viral pneumonia, e.g. coronavirus pneumonia, such as COVID-19 pneumonia) in a patient comprising:
a. administering a first weight-based 8 mg/kg intravenous dose of tocilizumab to the patient; and
b. further comprising administering a second weight-based 8 mg/kg intravenous dose of tocilizumab to the patient 8-12 hours after the first dose (e.g. 8-11 hours after the first dose) or 8-24 hours, wherein the patient experiences no improvement or ≥ one-category worsening on an ordinal scale of clinical status following the first dose.

In another embodiment, the invention provides a method of treating pneumonia (including viral pneumonia, e.g. coronavirus pneumonia, such as COVID-19 pneumonia) in a patient comprising administering an IL6 antagonist to the patient in an amount effective to achieve a greater improvement in clinical outcome than standard of care (SOC) as measured on an ordinal scale of clinical status.

In one embodiment, the IL6 antagonist binds IL6 receptor.

In one embodiment, the IL6 antagonist is tocilizumab, sarliumab, satralizumab, and/or TZLS-501.

In another embodiment of any of the methods herein, one may treat the patient with SOC along with the IL6 antagonist. SOC includes, for example, supportive care, anti-viral agent(s), and/or low-dose corticosteroid(s) as disclosed above.

In another embodiment, the invention provides a method of treating acute respiratory distress syndrome (ARDS) in a patient who does not have elevated IL6 level comprising administering an IL6 antagonist (e.g. an IL6 receptor antibody such as tocilizumab) to the patient. The patient with ARDS may have viral pneumonia, e.g. COVID-19 pneumonia.

These additional drugs as set forth herein are generally used in the same dosages and with administration routes as used hereinbefore or about from 1 to 99% of the heretofore-employed dosages. If such additional drugs are used at all, preferably, they are used in lower amounts than if the first medicament were not present, especially in subsequent dosings beyond the initial dosing with the first medicament, so as to eliminate or reduce side effects caused thereby.

The combined administration of an additional drug includes co-administration (concurrent administration), using separate formulations or a single pharmaceutical formulation, and consecutive administration in either order, wherein preferably there is a time period while both (or all) active agents (medicaments) simultaneously exert their biological activities.

### V. Articles of Manufacture

In another embodiment of the invention, articles of manufacture containing materials useful for the treatment pneumonia (including viral pneumonia, e.g. coronavirus pneumonia, such as COVID-19 pneumonia) and/or acute respiratory distress syndrome (ARDS) described above are provided.

The article of manufacture optionally further comprises a package insert with instructions for treating pneumonia (including viral pneumonia, e.g. coronavirus pneumonia, such as COVID-19 pneumonia) and/or acute respiratory distress syndrome (ARDS) in a subject, wherein the instructions indicate that treatment with the antibody as disclosed herein treats the pneumonia (e.g. including viral pneumonia, e.g. coronavirus pneumonia, such as COVID-19 pneumonia) and/or the acute respiratory distress syndrome (ARDS).

Further details of the invention are illustrated by the following non-limiting Example. The disclosures of all citations in the specification are expressly incorporated herein by reference.

### Example 1: A Randomized, Double-Blind, Placebo-Controlled, Multicenter Study to Evaluate the Safety and Efficacy of Tocilizumab in Patients with Severe Covid-19 Pneumonia

This is a Phase III, randomized, double-blind, placebo-controlled, multicenter study to assess the efficacy and safety of TCZ in combination with SOC compared with matching placebo in combination with SOC in hospitalized adult patients with severe COVID-19 pneumonia. Approximately 330 patients that have been diagnosed with COVID-19 pneumonia and meet the entry criteria in centers will be treated. Specific objectives and corresponding endpoints for the study are outlined below.

### Efficacy Objectives

### Primary Efficacy Objective

The primary efficacy objective for this study is to evaluate the efficacy of TCZ compared with placebo in combination with SOC for the treatment of severe COVID-19 pneumonia on the basis of the following endpoint:

1. Clinical status assessed using a 7-category ordinal scale at Day 28

### Secondary Efficacy Objectives

The secondary efficacy objective for this study is to evaluate the efficacy of TCZ compared with placebo in combination with SOC for the treatment of severe COVID-19 pneumonia on the basis of the following endpoints:
1. Time to clinical improvement (TTCI) defined as a National Early Warning Score 2 (NEWS2) of ≤ 2 maintained for 24 hours
2. Time to improvement of at least 2 categories relative to baseline on a 7-category ordinal scale of clinical status
3. Incidence of mechanical ventilation
4. Ventilator-free days to Day 28
5. Organ failure-free days to Day 28
6. Incidence of intensive care unit (ICU) stay
7. Duration of ICU stay
8. Time to clinical failure, defined as the time to death, mechanical ventilation, ICU admission, or withdrawal (whichever occurs first)
9. Mortality rate at Days 7, 14, 21, 28, and 60
10. Time to hospital discharge or “ready for discharge” (as evidenced by normal body temperature and respiratory rate, and stable oxygen saturation on ambient air or ≤ 2L supplemental oxygen)
11. Duration of supplemental oxygen

### Additional Efficacy Objective

The further efficacy objective for this study is to evaluate the efficacy of TCZ compared with placebo in combination with SOC for the treatment of severe COVID-19 pneumonia on the basis of the following endpoints:
1. Incidence of vasopressor use
2. Duration of vasopressor use
3. Incidence of extracorporeal membrane oxygenation (ECMO)
4. Duration of ECMO

### Safety Objective

The safety objective for this study is to evaluate the safety of TCZ compared with placebo in combination with SOC for the treatment of severe COVID-19 pneumonia on the basis of the following endpoints:
1. Incidence and severity of adverse events, with severity determined according to National Cancer Institute Common Terminology Criteria for Adverse Events (NCI CTCAE) v5.0
2. COVID-19 (SARS-CoV-2) viral load over time, as collected by nasopharyngeal swab and bronchoalveolar lavage (BAL) samples (if applicable)
3. Time to reverse-transcriptase polymerase chain reaction (RT-PCR) virus negativity
4. The proportion of patients with any post-treatment infection
5. Change from baseline in targeted clinical laboratory test results

### Pharmacodynamic Objective

The pharmacodynamic objective for this study is to characterize the pharmacodynamic effects of TCZ in patients with COVID-19 pneumonia via longitudinal measures of the following analytes relative to baseline:

1. Serum concentrations of IL-6, sIL-6R, ferritin, and CRP at specified timepoints

### Pharmacokinetic Objective

The PK objective for this study is to characterize the TCZ PK profile in patients with COVID-19 pneumonia on the basis of the following endpoint:

1. Serum concentration of TCZ at specified timepoints

### Description of the Study

Patients must be at least 18 years of age with confirmed COVID-19 infection per WHO criteria, including a positive PCR of any specimen (e.g., respiratory, blood, urine, stool, other bodily fluid). At the time of enrollment, patients must have SpO₂ ≤ 93% or PaO₂/FiO₂ < 300 mmHg despite being on SOC, which may include anti-viral treatment, low dose steroids, and supportive care.

Patients in whom, in the opinion of the treating physician, progression to death is imminent and inevitable within the next 24 hours, irrespective of the provision of treatments, will be excluded from the study. Patients with active tuberculosis (TB) or suspected active bacterial, fungal, viral, or other infection (besides COVID-19) will be excluded from the study.

Patients will be randomized as soon as possible after screening at a 2:1 ratio to receive blinded treatment of either TCZ or a matching placebo, respectively. Study treatment must be given in combination with SOC. The randomization will be stratified by geographic region (North America, Europe, and other) and mechanical ventilation (yes, no).

Patients assigned to the TCZ arm will receive one infusion of TCZ 8 mg/kg, with a maximum dose of 800 mg, and patients assigned to the placebo arm will receive one infusion of placebo both in addition to SOC.

For both arms, if the clinical signs or symptoms worsen or do not improve (e.g. reflected by sustained fever or at least a one-category worsening on the 7-category ordinal scale of clinical status), one additional infusion (8 m/kg with maximum dose of 800 mg) of blinded treatment of TCZ or placebo can be given, 8-12 hours (or 8-24 hours) after the initial infusion.

### After Day 28

Patients will be followed up for a total of 60 days after first dose of study medication.

For patients who are discharged, between Day 28 and study completion visits may be conducted via telephone.

During the study, standard supportive care will be given according to clinical practice.

Patients will be followed-up for a period of 60 days starting from the randomization.

### Control Group

The study will compare the efficacy and safety of TCZ IV with matching placebo in combination with SOC. Despite the lack of targeted treatments for COVID-19, SOC for patients with severe COVID-19 pneumonia generally includes supportive care and may include available anti-viral agents and low-dose corticosteroids as dictated by local treatment guidelines.

### Patients

This study aims to enroll approximately 330 hospitalized patients with severe COVID-19 pneumonia.

### Inclusion Criteria

Patients must meet the following criteria for study entry:
1. Age ≥ 18 years
2. Hospitalized with COVID-19 pneumonia confirmed per WHO criteria (including a positive PCR of any specimen; e.g., respiratory, blood, urine, stool, other bodily fluid) and evidenced by chest X-ray or CT scan
3. SpO₂ ≤ 93% or PaO2/FiO₂ < 300 mmHg

### Exclusion Criteria

Patients who meet any of the following criteria will be excluded from study entry:
1. Known severe allergic reactions to TCZ or other monoclonal antibodies
2. Active TB infection
3. Suspected active bacterial, fungal, viral, or other infection (besides COVID-19)
4. In the opinion of the investigator, progression to death is imminent and inevitable within the next 24 hours, irrespective of the provision of treatments
5. Have received oral anti-rejection or immunomodulatory drugs (including TCZ) with the past 6 months
6. Participating in other drug clinical trials (participation in COVID-19 anti-viral trials may be permitted if approved by Medical Monitor)
7. ALT or AST > 10 x ULN detected within 24 hours at screening or at baseline (according to local lab oratory reference ranges)
8. ANC < 1000/µL at screening and baseline (according to local laboratory reference ranges)
9. Platelet count < 50,000/µL at screening and baseline (according to local laboratory reference ranges)
10. Pregnant or breastfeeding, or positive pregnancy test in a pre-dose examination
11. Treatment with an investigational drug within 5 half-lives or 30 days (whichever is longer) of randomization (investigational COVID-19 antivirals may be permitted if approved by Medial Monitor)
12. Any serious medical condition or abnormality of clinical laboratory tests that, in the investigator’s judgment, precludes the patient’s safe participation in and completion of the study

### 7-Category Ordinal Scale

Assessment of clinical status using a 7-category ordinal scale will be recorded at baseline on Day 1 and then again once daily every morning (between 8 am and 12 pm) while hospitalized. The ordinal scale categories are as follows:
1. Discharged (or “ready for discharge” as evidenced by normal body temperature and respiratory rate, and stable oxygen saturation on ambient air or ≤ 2 L supplemental oxygen)
2. Non-ICU hospital ward (or “ready for hospital ward”) not requiring supplemental oxygen
3. Non-ICU hospital ward (or “ready for hospital ward”) requiring supplemental oxygen
4. ICU or non-ICU hospital ward, requiring non-invasive ventilation or high-flow oxygen
5. ICU, requiring intubation and mechanical ventilation
6. ICU, requiring ECMO or mechanical ventilation and additional organ support (e.g. vasopressors, renal replacement therapy)
7. Death

In general, patients with oxygen saturation consistently ≤ 90% should be considered for escalation to a higher clinical status category, while patients with oxygen saturation consistently ≥ 96% should be considered for de-escalation to a lower category. Patients on supplemental oxygen should be evaluated at least daily and considered for reduction or discontinuation of oxygen support. Actual changes in level of support will be at the discretion of the clinician(s) treating the patient based on the patient’s overall condition and may be dictated by other clinical and non-clinical considerations.

Normal body temperature is defined as oral, rectal, or tympanic temperature 36.1-38.0° C. Normal respiratory rate is defined as 12-20 breaths per minute.

### National Early Warning Score (NEWS) 2

The NEWS2 score is disclosed in Royal College of Physicians. National early warning score (NEWS) 2. Standardizing the assessment of acute-illness severity in the NHS. London: RCP (2017).

It involves evaluating the following parameters.

| | | | | | | | |
|---|---|---|---|---|---|---|---|
| Physiological Parameter Score | 3 | 2 | 1 | 0 | 1 | 2 | 3 |
| Respiration rate (per Minute) | ≤8 | | 9-11 | 12-20 | | 21-24 | ≥25 |
| SpO₂ Scale 1 (%) | ≤91 | 92-93 | 94-5 | ≥96 | | | |
| SpO₂ Scale 2 (%) | ≤83 | 84-85 | 86-87 | 88-92 ≥93 on | 93-94 on oxygen | 95-96 on oxygen | ≥97 on oxygen |
| | | | | air | | | |
| Air or Oxygen? | | Oxygen | | Air | | | |
| Systolic Blood Pressure (mmHg) | ≤90 | 91-100 | 101-100 | 111-219 | | | ≥220 |
| Pulse (Per minute) | ≤40 | | 41-50 | 51-90 | 91-110 | 111-130 | ≥131 |
| Consciousness | | | | Alert | | | CVPU |
| Temperature (°C) | ≤35.0 | | 35.1-36.0 | 36.1-38.0 | 38.1-39.0 | ≥39.1 | |

SpO₂ = oxygen saturation; CVPU = confusion, voice, pain, unconsciousness.

The oxygen saturation should be scored according to either the SpO₂ Scale 1 or 2 presented in the table above. The SpO₂ Scale 2 is for patients with a target oxygen saturation requirement of 88%-92% (e.g., in patients with hypercapnic respiratory failure related to advanced lung diseases, such as chronic obstructive pulmonary disease [COPD]). This should only be used in patients confirmed to have hypercapnic respiratory failure by blood gas analysis on either a prior or their current hospital admission.

The decision to use the SpO₂ Scale 2 should be made by the treating physician and should be recorded in the eCRF. In all other circumstances, the SpO₂ Scale 1 should be used.

For physiological parameter “Air or Oxygen?”: Any patients requiring the use of oxygen or other forms of ventilation to maintain oxygen saturations and support respiration should be assigned a score of 2.

The consciousness level should be recorded according to the best clinical condition of the patient during the assessment. Patients who are assessed as “Alert” (A) should be assigned a score of 0. Patients assessed as “New Confusion” (C), “Responsive to Voice” (V), “Responsive to Pain” (P), or “Unconscious” should be assigned a score of 3.

Scores should be assigned for respiratory rate, systolic blood pressure, pulse, and temperature according to the table above.

NEWS2 values will be calculated electronically throughout the study by the Sponsor based upon entry of vital sign parameters by the investigator in the appropriate eCRF.

### Example Case Calculation

An 82-year-old lady was admitted, tested positive to COVID-19 and admitted to high dependency unit for non-invasive ventilation. Her taken observations and corresponding NEWS2 score are as follows:

| Physiological Parameter | Observation | Component Score |
|---|---|---|
| Respiratory rate (per min) | 26 | 3 |
| Oxygen saturation (SpO₂ %) | 95% | 1 |
| Supplemental Oxygen | Yes | 2 |
| Systolic blood pressure (mmHg) | 95 | 2 |
| Pulse Rate (bpm) | 109 | 1 |
| Conscious level | New confusion | 3 |
| Temperature (°C) | 39 | 1 |
| | Total NEWS2 Score | 13 |

### Liver Function

Patients should be assessed for liver function prior to each dose of TCZ or matching placebo on Day 1. In clinical trials, mild and moderate elevations of hepatic transaminases have been observed with TCZ treatment. Recommended TCZ dose modifications for elevated liver enzymes in these populations are not applicable to this study due to single dose therapy (with possible additional infusion) with TCZ or placebo. The finding of an elevated ALT or AST (> 3 x ULN) in combination with either an elevated total bilirubin (> 2 x ULN) or clinical jaundice in the absence of cholestasis or other causes of hyperbilirubinemia is considered to be an indicator of severe liver injury (as defined by Hy’s Law). Adverse event the occurrence of either of the following can be reported:
1. Treatment-emergent ALT or AST > 3 x ULN in combination with total bilirubin > 2 × ULN
2. Treatment-emergent ALT or AST > 3 × ULN in combination with clinical jaundice

### Results and Conclusions

It is anticipated that the treatment herein with weight-based (8 mg/kg ≤ 800 mg) intravenous dose of tocilizumab, with optional single second weight-based (8 mg/kg ≤ 800 mg) dose of tocilizumab 8-12 hours (including 8-11 hours) or 8-24 hours after the initial dose (if the patient’s clinical signs or symptoms do not improve or worsen as reflected by at least a one-category worsening on an ordinal scale of clinical status) will achieve any one or more of the primary, secondary, or additional endpoints, while having acceptable toxicity according to the safety endpoints specified herein.

### Example 2: Tocilizumab and Remdesivir Combination Therapy For COVID-19 Pneumonia

This is a randomized, double-blind, double-dummy study of about 450 patients with 3 arms randomized 4:1:1 to:
Arm A: TCZ plus RDV+SOC
Arm B: TCZ+SOC
Arm C: RDV+SOC

TCZ arm will be administered 8 mg/kg (with a maximum dose of 800 mg) and, if the patient’s clinical signs or symptoms worsen or do not improve (e.g. reflected by sustained fever or at least a one-category worsening on the 7-category ordinal scale of clinical status), one additional infusion of blinded treatment of TCZ (8 mg/kg, with a maximum dose of 800 mg) can be given 8-24 hours after the initial infusion.

RDV is administered 200 mg on Day 1 followed by RDV 100 mg on Days 2, 3, 4, and 5 or RDV 200 mg on Day 1 followed by RDV 100 mg on Days 2, 3, 4, 5, 6, 7, 8, 9, and 10.

SOC for patients with severe COVID-19 pneumonia generally includes supportive care and may include anti-viral agents other than RDV (preferably only one other anti-viral treatment) and low-dose corticosteroids as dictated by local treatment guidelines.

The “Inclusion Criterion” and “Exclusion Criteria” are as described above for Example 1.

The Efficacy and Safety Objectives are as described in Example 1.

It is anticipated that the combination treatment with TCZ and RDV will achieve any one or more of the primary, secondary, or additional endpoints, while having acceptable toxicity. It is further anticipated that the combination treatment with TCZ+RDV+SOC will more effectively treat COVID-19 pneumonia than TCZ+SOC (i.e. without RDV) and RDV+SOC (i.e. without TCZ).

What is claimed is:

1. A method of treating viral pneumonia in a patient comprising administering an effective amount of a combination of tocilizumab and remdesiver to the patient.

2. The method of claim 1, wherein the viral pneumonia is COVID-19 pneumonia.

3. The method of claim 2, wherein the viral pneumonia is severe pneumonia.

4. The method of claim 3, wherein the viral pneumonia is severe COVID-19 pneumona.

5. The method of claim any one of the preceding claims, further comprising treating the patient with COVID-19 standard of care (SOC).

6. The method of claim 5, wherein the SOC comprises one or more of: supportive care, antiviral agent other than remdisivir, and low-dose corticosteroid.

7. The method of claim any one of the preceding claims, wherein the effective amount of tocilizumab comprises a first weight-based 8 mg/kg intravenous dose of tocilizumab optionally followed by a second weight-based 8 mg/kg intravenous dose of tocilizumab 8-24 hours after the first dose.

8. The method of any one of the preceding claims, wherein the effective amount of remdesivir comprises an initial one-time dose of 200 mg followed by 100 mg per day.

9. The method of claim 8, comprising 5 to 10 total doses of remdesivir.

10. The method of any one of the preceding claims, wherein the effective amount of the combination is more effective at treating the viral pneumonia than tocilizumab without remdesivir and than remdesevir without tocilizumab.

11. The method of claim 7, wherein the second weight-based intravenous dose of tocilizumab is administered to the patient 8-24 hours after the first dose.
